(19) United States
(12) Patent Application Publication
(10) Pub. No.: US 2008/0193534 A1
(43) Pub. Date: Aug. 14, 2008

(54) Process

(75) Inventors: Lennart Lindfors, Molndal (SE); Urban Skantze, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/063,438

(22) PCT Filed: Aug. 9, 2006
(86) PCT No.: PCT/SE2006/000933

§ 371 Date: Feb. 11, 2008

(30) Foreign Application Priority Data
Aug. 12, 2005 (SE) 0501807-2

(51) Int. Cl. A61K 9/107

(52) U.S. Cl. 424/484

(57) ABSTRACT

The present invention relates to a process for the preparation of a stable dispersion of particles, particularly sub-micron particles in an aqueous medium and to a stable dispersion of particles in a liquid medium. The process provided comprises the following steps: 1) combining a) an emulsion comprising a continuous aqueous phase; an inhibitor; a stabiliser; with b) the substantially water-insoluble substance; and 2) increasing the temperature to vicinity of the melting temperature of the substantially water-insoluble substance. The sub-micron dispersion provided exhibit reduced or substantially no particle growth during storage and reduced crystallisation rate of the substantially water insoluble active compound.

The present invention relates to a process for the preparation of a stable dispersion of particles, particularly sub-micron particles in an aqueous medium and to a stable dispersion of particles in a liquid medium. More particularly the present invention relates to a process for the preparation of a dispersion of particles comprising an amorphous substantially water-insoluble pharmacologically active compound of a high concentration in an aqueous medium, which exhibit reduced crystallisation rate of the substantially water insoluble active compound. Further, the particles exhibit substantially no increase in size upon storage in the aqueous medium, in particular aqueous dispersions of particles that exhibit substantially no particle growth mediated by Ostwald ripening.

Dispersions of a solid material in a liquid medium are required for a number of different applications including paints, inks, dispersions of pesticides and other agrochemicals, dispersions of biocides and dispersions of pharmacologically active compounds. In the pharmaceutical field many pharmacologically active compounds have very low aqueous solubility, which can result in low bioavailability. The bioavailability of such compounds may be improved by reducing the particle size of the compound, particularly to a sub-micron size, because this improves dissolution rate and hence absorption of the compound. This effect is expected to be even more pronounced using amorphous particles.

The formulation of a pharmacologically active compound as an aqueous suspension, particularly a suspension with a sub-micron particle size, enables the compound to be administered intravenously and thereby providing an alternative route of administration which may increase bioavailability compared to oral administration.

However, there will generally be a differential rate of dissolution if there is a range of particles sizes dispersed in a medium. The differential dissolution rate has an impact on the thermodynamical stability, the smaller particles are thermodynamically unstable relative to the larger particles. This gives rise to a flux of material from the smaller particles to the larger particles. The effect is that the smaller particles dissolve in the medium, whilst material is deposited onto the larger particles thereby giving an increase in particle size. One such mechanism for particle growth is known as Ostwald ripening (Ostwald, Z Phys. Chem. (34), 1900, 495-503). The growth of particles in a dispersion can result in instability of the dispersion during storage due to the sedimentation of particles from the dispersion. It is particularly important that the particle size in a dispersion of a pharmacologically active compound remains constant because a change in particle size is likely to affect the bioavailability and hence the efficacy of the compound. Furthermore, if the dispersion is to be used for intravenous administration, growth of the particles in the dispersion may render the dispersion unsuitable for this purpose.

Theoretically particle growth resulting from Ostwald ripening would be eliminated if all the particles in the dispersion were the same size. However, in practice, it is not possible to achieve a completely uniform particle size and even small differences in particle sizes can give rise to particle growth.

Aqueous suspensions of a solid material can be prepared by mechanical fragmentation, for example by milling. U.S. Pat. No. 5,145,684 describes wet milling of a suspension of a sparingly soluble compound in an aqueous medium. However, a major disadvantage using wet milling is contamination from the beads used in the process. Furthermore, mechanical fragmentation is less efficient in terms of particle size reduction when applied to non-crystalline starting material.

U.S. Pat. No. 4,826,689 describes a process for the preparation of uniform sized particles of a solid by infusing an aqueous precipitating liquid into a solution of the solid in an organic liquid under control of temperature and infusion rate, thereby controlling the particle size.

U.S. Pat. No. 4,997,454 describes a similar process in which the precipitating liquid is non-aqueous. However, when the particles have a small but significant solubility in the precipitating medium particle size growth is observed after the particles have been precipitated. To maintain a particular particle size using these processes it is necessary to isolate the particles as soon as they have been precipitated to minimise particle growth. Consequently, particles prepared according to these processes cannot be stored in a liquid medium as a dispersion. Furthermore, for some materials the rate of Ostwald ripening is so rapid that it is not practical to isolate small particles (especially nano-particles) from the suspension.

U.S. Pat. No. 5,100,591 describes a process for preparing particles, comprising a complex between a water insoluble substance and a phospholipids, are prepared by co-precipitation of the substance and phospholipid into an aqueous medium. Generally the molar ratio of phospholipid to substance is 1:1 to ensure that a complex is formed.

U.S. Pat. No. 6,197,349 describes a process for the formation of amorphous particles by melting a crystalline compound and mixing the compound with a stabilising agent, e.g. a phospholipid, and dispersing this mixture in water at elevated temperature using high pressure homogenization, after which the temperature is lowered to e.g. ambient temperature.

WO 03/059319 describes the formation of small particles by adding a solution of a drug dissolved in a water immiscible organic solvent to a template oil-in-water emulsion after which the water immiscible organic solvent is evaporated off. Water is then removed, e.g. using a spray-drying process to obtain a powder.

U.S. Pat. No. 5,700,471 describes a process for producing small amorphous particles in which crystalline material dispersed in water, is heated and subjected to turbulent mixing above the melting temperature, and the resulting melt emulsion is immediately spray-dried or converted into a suspension by cooling. However, such suspensions will exhibit particle growth mediated by Ostwald ripening. Furthermore, according to U.S. Pat. No. 5,700,471 some substances are not amenable to such a process without using an additional organic solvent due to particle agglomeration. One such compound is fenofibrate.

WO 03/013472 describes a precipitation process. This is a precipitation process without the need of water immiscible solvents for the formation of dispersions of amorphous nanoparticles. The dispersion prepared herein exhibit little or no particle growth after precipitation mediated by Ostwald ripening.

We have surprisingly found that stable dispersions of amorphous sub-micron particles may be prepared by a process where a substantially water-insoluble substance is mixed with a continuous aqueous phase comprising a component inhibiting the Ostwald ripening, i.e. “the inhibitor”, and the mixture obtained is treated for allowing the substantially water insoluble substance to migrate into the oily phase formed by the inhibitor. Thus the process according to the invention is without precipitation which is advantageous when working in larger scales.

The inhibitor with the said property is suitable also completely miscible with the amorphous phase of the substantially water-insoluble substance formed when the substance is heated. The ratio of water insoluble substance to inhibitor is less than 10:1 (w/w). The mixture is then heated to the vicinity of the melting point of the substantially water insoluble substance for a short period of time, after which the mixture is cooled to ambient temperature. The dispersion obtained comprises sub-micron particles having a high concentration of the substantially water-insoluble substance. Since the process described is not a precipitation process high concentrations can be obtained in aqueous systems (Vitale et al., Langmuir 19, 4105 (2003)).

The Process

The process according to the present invention enables stable dispersions of very small amorphous particles, especially particles having a diameter of below 500 nm, to be prepared at high concentrations without the need to quickly isolate the particles from the liquid medium to reduce particle growth and crystallisation rate. The dispersion of sub-micron particles obtainable by the process may be ready for use. However, optionally, the particles may be recovered from the dispersion. Suitable methods for removing the aqueous phase are for example evaporation, spray-drying, spray-granulation, freeze-granulation or lyophilisation. The dispersion may also be concentrated by removing excess water from the dispersion, for example by heating the dispersion under vacuum, reverse osmosis, dialysis, ultra-filtration or cross-flow filtration.

According to one aspect of the present invention there is provided a process for the preparation of a stable dispersion of amorphous particles of sub-micron size in an aqueous medium. The process comprises the following steps:

1) combining

a) an emulsion comprising
a continuous aqueous phase;
an inhibitor;
a stabiliser;
   with

b) a substantially water-insoluble substance in the crystalline state; and

2) increasing the temperature of the mixture to the vicinity of the melting temperature of the substantially water-insoluble substance.

The mixture may then, during step 2) be kept at this temperature until the substantially water insoluble substance in crystalline state form is melted and thus transferred into amorphous state. The temperature is then lowered, for example, to ambient temperature, and the dispersion of amorphous sub-micron particles is obtained.

For substances with melting points above 100° C., the process is performed under pressure, e.g. using a high pressure reactor, due to the boiling point of the aqueous medium.

The particles, i.e. the “sub-micron particles”, obtained by the method of the invention have a mean particle size of less than 10 μm, for example less than 5 μm, or less than 1 μm or even less than 500 nm. It is especially preferred that the particles in the dispersion have a mean particle size of from 10 to 500 nm, for example from 50 to 300 nm, or from 100 to 200 nm. The mean size of the particles may be measured using conventional techniques, for example by dynamic light scattering, to obtain the intensity averaged particle size.

Amorphous particles will eventually revert to a thermodynamically more stable crystalline form upon storage as an aqueous dispersion. The time required for such particles to crystallise is dependent upon the components of the particles and the dispersion of the pharmaceutically active compound and may vary from a few hours to a number of weeks. Generally such re-crystallisation will also result in particle growth. The formation of larger crystalline particles is unsuitable for pharmaceutical administration and they are also prone to sedimentation from the dispersion. The conversion of the amorphous substance to crystalline substance by crystal nucleation and growth is generally difficult to control. However, according to the present invention, completely miscible amorphous drug/inhibitor systems, enables not only a possibility to influence crystal nucleation but also a reduced crystal growth rate. These advantages are obtained by having a ratio of water-insoluble substance to inhibitor below 10:1 (w/w), for example 4:1, or 2:1 (w/w).

The sub-micron dispersion obtained by the process of the invention is stable, by which we mean that the particles in the dispersion exhibit reduced or substantially no particle growth mediated by Ostwald ripening, as well as that the particles are kept amorphous during storage. The amorphous substance exhibit reduced or substantially no crystallization and the sub-micron dispersion can be stable in the meaning of remaining in the amorphous state during a considerable long time, i.e. the crystallization rate can be reduced significantly.

By the term “reduced or substantially no crystallisation” is meant that the rate of crystallization in the obtained amorphous dispersions is reduced by the use of a higher inhibitor/drug ratio compared to particles prepared using a lower inhibitor/drug ratio.

By the term “reduced particle growth” is meant that the rate of particle growth mediated by Ostwald ripening is reduced compared to particles prepared without the use of an inhibitor. By the term “substantially no particle growth” is meant that the mean size of the particles in the aqueous medium does not increase by more than 10%, for example not more than 5%, over a period of 1 hour at ambient temperature after the formation according to the present process. Preferably the particles exhibit substantially no particle growth.

The presence of the inhibitor together with the water-insoluble substance significantly reduces or eliminates particle growth mediated by Ostwald ripening, as hereinbefore described.

When the emulsion and the substantially water-insoluble substance is mixed and the temperature is increased as described as step 2) of the process, the substantially water-insoluble substance is transported to the phase comprising the inhibitor, which requires that the inhibitor is completely miscible with the amorphous phase of the substantially water-insoluble substance.

To achieve the improved stability of the amorphous submicron particles all crystalline material is transferred to the amorphous state. This is performed by increasing the temperature in step 2) to the vicinity of the melting temperature of the substantially water-insoluble substance, for example suitable to a temperature of ±20° C. of its melting point, or ±15° C. of its melting point, or ±10° C. of its melting point, or ±5° C. of its melting point. In case that not all crystalline material is transferred into amorphous state the remaining crystalline material may act as seeds for crystallisation.

The process according to the present invention enables stable dispersions of very small particles, especially submicron particles, to be prepared at high concentration without the need to quickly isolate the particles form the liquid medium to prevent particle growth. With “high concentration” is here meant between 1 to 30% by weight of the total concentration of the substantially water-insoluble substances in the dispersion of the invention, for example 5, 10, 15, 20 or 25% by weight. As said before, the amorphous particles may exhibit crystallisation i.e. the amorphous substance in the particles formed may be transferred from amorphous state to crystalline state, a process which is due to thermodynamic rules. However, the rate of this thermodynamically determined process may be lowered by decreasing the ratio of water-insoluble substance to inhibitor being below 10:1 (w/w), for example 9:1, 8:1, 7:1, 6:1, 5:1, 4:1, 3:1, 2:1, or 1:1 (w/w). By decreasing this ratio, the bulk concentration, i.e. the amorphous solubility, in the dispersion of amorphous submicron particles can be lowered. The amorphous solubility in, for example, water may be determined by measuring static light scattering as a function of dilution of the amorphous suspension of the water-insoluble substance by adding small volumes of the amorphous dispersion of water-insoluble substance successively to a fluorescence cuvette containing water to give the desired concentrations. The optimal ratio is depending upon the water-insoluble substance and the inhibitor or inhibitor/co-inhibitor selected.

The invention also provides a process where particles of the same size are obtained even when the concentration of the water-insoluble substance varies between the particles. Such particles are obtained in the present process as the formation of particles according to the present invention is independent nucleation, and differs from precipitation type processes.

The Water-Insoluble Substance

In one embodiment of the invention, the emulsion is mixed with the particles of water-insoluble substance which being initially in a crystalline state. These crystalline particles may be of any size of 1 μm or above, for example between 1 μm and 500 μm or between 1 μm and 200 μm.

In one embodiment the crystalline particles of water-insoluble substance are first prepared as a suspension in an aqueous phase, optionally containing one or more stabilisers, optionally the stabiliser may also be in combination with other water-miscible solvents.

The aqueous phase may consist of water, or of water in mixture of one or more water miscible organic solvents.

As will be understood, the selection of water-miscible organic solvent will be dependent upon the nature of the substantially water-insoluble substance. Examples of such water-miscible solvents include water-miscible alcohol, for example methanol, ethanol, n-propyl alcohol, isopropyl alcohol, tert-butyl alcohol, ethylene glycol; dimethylsulfoxide, a water-miscible ether, for example tetrahydrofuran, a water-miscible nitrile, for example, acetonitrile; a water-miscible ketone, for example acetone or methyl ethyl ketone; an amide, for example dimethylacetamide, dimethylformamide, or a mixture of two or more of the above mentioned water-miscible organic solvents. Preferred water-miscible organic solvents are ethanol, dimethylsulfoxide, dimethylacetamide.

In one embodiment, the water insoluble substance is added to the emulsion in an amorphous form. The water-insoluble substance in amorphous form may be obtained, for example, by spray-drying, spray-freezing, freeze-drying or spray-granulation. This list of methods for drying is non-exhaustive. Furthermore, the process of the invention is also suitable for amorphous substances not available in crystalline state.

The substantially water-insoluble substance is preferably a substantially water-insoluble organic substance. By “substantially water insoluble” is meant a substance that has solubility in water at 25° C. of less than 0.5 mg/ml, preferably less than 0.1 mg/ml and especially less than 0.05 mg/ml.

The greatest effect on Ostwald ripening inhibition is observed when the substance has solubility in water at 25° C. of more than 0.05 μg/ml. In a preferred embodiment the substance has a solubility in the range of from 0.005 μg/ml to 0.5 mg/ml, for example from 0.05 μg/ml to 0.05 mg/ml.

The solubility of the substance in the crystalline state in water may be measured using a conventional technique. For example, a saturated solution of the substance is prepared by adding an excess amount of the substance to water at 25° C. and allowing the solution to equilibrate for 48 hours. Excess solids are removed by centrifugation or filtration and the concentration of the substance in water is determined by a suitable analytical technique such as HPLC.

By the invention, a process for producing sub-micron particles comprising a substantially water-insoluble substance having a melting point of up to 300° C. is provided. For example the substantially water insoluble substance has a melting point below 250° C., such as below 200° C., or below 175° C., such as 150° C.

The process according to the present invention may be used to prepare stable aqueous dispersions of a wide range of substantially water-insoluble substances. Suitable substances include but are not limited to pigments, pesticides, herbicides, fumgicides, industrial biocides, cosmetics, pharmacologically active compounds and pharmacologically inert substances such as pharmaceutically acceptable carriers and diluents.

In a preferred embodiment the substantially water-insoluble substance is a substantially water-insoluble pharmacologically active substance. Numerous classes of pharmacologically active compounds are suitable for use in the present invention including but not limited to substantially water-insoluble anti-cancer agents (for example bicalutamide), steroids, preferably glucocorticosteroids (especially anti-inflammatory glucocorticosteroids, for example budesonide) antihypertensive agents (for example felodipine or prazosin), beta-blockers (for example pindolol or propranolol), hypolipidaemic agents (for example fenofibrate), aniticoagulants, antithrombotics, antifungal agents (for example griseofulvin), antiviral agents, antibiotics, antibacterial agents (for example ciprofloxacin), antipsychotic agents, antidepressants, sedatives, anaesthetics, anti-inflammatory agents (including compounds for the treatment of gastrointestinal inflammatory diseases, for example compounds described in WO99/55706 and other anti-inflammatory compounds, for example ketoprofen), antihistamines, hormones (for example testosterone), immunomodifiers, or contraceptive agents. The substance may comprise a single substantially water-insoluble substance or a combination of two or more such substances.

The Emulsion

The emulsion of the present invention is an emulsion comprising a continuous aqueous phase and an oil phase constituted by the inhibitor, i.e. when water is chosen as the continuous aqueous phase, an oil-in-water emulsion. When water, or water in admixture with a water-miscible solvent, is used in the process according to the invention, an emulsion comprising the inhibitor is formed. The emulsion is an oil-in-water emulsion. The emulsion may also comprise further components as defined below.

The emulsion is produced by conventional methods, for example, the inhibitor, a stabilizer and water forms a mixture before it is then homogenised. The homogenisation is performed, for instance, by sonication or high-pressure homogenisation.

Preferably, the process of the invention is an aqueous based process wherein the continuous aqueous consists of water. However, also other options for the continuous aqueous phase are possible, for example, water mixed with a water-miscible solvent. The water miscible solvent may be chosen from the list above or mixture thereof. Further, other options for the aqueous phase may be mixtures of water and low molecular sugars. Such components are added in order to promote the conversion of the amorphous suspension to the dry state e.g. by lyophilisation, spray-drying or spray-granulation. Preferably, water is used for the process according to the invention. The use of water is an important aspect from an environmental perspective. A water-based process is also advantageous as traces of organic solvent in the particles can be avoided.

The Stabiliser

The emulsion also comprises at least one stabiliser which prevent aggregation of the emulsion droplets. In a similar way the amorphous particles tend to aggregate in the final dispersion unless a stabiliser is present.

Stabilisers suitable for the prevention of particle aggregation in dispersions are well known to those skilled in the art. Suitable stabilisers include dispersants and surfactants (which may be anionic, cationic or non-ionic) or a combination thereof. Suitable dispersants include, a polymeric dispersant, for example a polyvinylpyrrolidone, a polyvinylalcohol or a cellulose derivative, for example hydroxypropylmethyl cellulose, hydroxy ethyl cellulose, ethylhydroxyethyl cellulose or carboxymethyl cellulose. Suitable anionic surfactants include alkyl and aryl sulphonates, sulphates or carboxylates, such as an alkali metal alkyl and aryl sulphonate or sulphate, for example, sodium dodecyl sulphate. Suitable cationic surfactants include quaternary ammonium compounds and fatty amines. Suitable non-ionic surfactants include, monoesters of sorbitan which may or may not contain a polyoxyethylene residue, ethers formed between fatty alcohols and polyoxyethylene glycols, polyoxyethylene-polypropylene glycols, an ethoxylated castor oil (for example Cremophor EL), ethoxylated hydrogenated castor oil, ethoxylated 12OH-stearic acid (for example Solutol HS15), phospholipids, for example phospholipids substituted by chains of polyethylene glycols (PEG). Examples are DPPE-PEG (dipalmitoyl phosphatidylethanolamine substituted with PEG2000 or PEG5000 or DSPE-PEG5000 (distearoyl phosphatidylethanolamine substituted by PEG5000). The stabiliser present in the aqueous phase may be a single stabiliser or a mixture of two or more stabilisers. In a preferred embodiment the aqueous phase contains a polymeric dispersant and a surfactant (preferably an anionic surfactant), for example a polyvinylpyrrolidone and sodium dodecyl sulphate. When the substantially water-insoluble material is a pharmacologically active compound it is preferred that the stabiliser is a pharmaceutically acceptable material.

Generally the aqueous phase will contain from 0.01 to 10% by weight, for example 0.01 to 5% by weight, preferably from 0.05 to 3% by weight and especially from 0.1 to 2% by weight of stabiliser.

The Inhibitor

Suitable for the present invention, the inhibitor fulfills the following:
the inhibitor is a compound that is substantially insoluble in water;
the inhibitor is less soluble in water than the substantially water-insoluble substance; and
the inhibitor is completely miscible with the amorphous phase of the substantially water-insoluble substance.

It is of importance for the present invention that the inhibitor affecting Ostwald ripening is completely miscible with the amorphous drug. As in WO 03/013472, the miscibility may be characterised by the Bragg-Williams interaction parameter χ. A value of χ being less than 2.5, more preferable χ less than 2 can characterize full miscibility between an amorphous drug and an Ostwald ripening inhibitor.

The inhibitor is a compound that is less soluble in water than the substantially water-insoluble substance present in the first solution. Preferably, the inhibitor is a hydrophobic organic compound. The inhibitor suitable for the process of the invention have an influence of the particle growth mediated by Ostwald ripening, as described in WO 03/013472.

Suitable inhibitors have water solubility at 25° C. of less than 0.1 mg/l, more preferably less than 0.01 mg/l. In an embodiment of the invention the solubility of the inhibitor in water at 25° C. is less than 0.05 μg/ml, for example from 0.1 ng/ml to 0.05 μg/ml.

In an embodiment of the invention the inhibitor has a molecular weight of less than 2000, for example less than 1000. In another embodiment of the invention the inhibitor has a molecular weight of less than 1000, for example less than 600. For example, the inhibitor may have a molecular weight in the range of from 200 to 2000, preferably a molecular weight in the range of from 400 to 1000, more preferably from 400 to 600.

Particular, suitable inhibitors include an inhibitor selected from classes (i) to (vi) described below, or a combination of two or more such inhibitors:

(i) a mono-, di- or (more preferably) a tri-glyceride of a fatty acid. Suitable fatty acids include medium chain fatty acids containing from 8 to 12, more preferably from 8 to 10 carbon atoms or long chain fatty acids containing more than 12 carbon atoms, for example from 14 to 20 carbon atoms, more preferably from 14 to 18 carbon atoms. The fatty acid may be saturated, unsaturated or a mixture of saturated and unsaturated acids. The fatty acid may optionally contain one or more hydroxyl groups, for example ricinoleic acid. The glyceride may be prepared by well known techniques, for example, esterifying glycerol with one or more long or medium chain fatty acids. In a preferred embodiment the inhibitor is a mixture of triglycerides obtainable by esterifying glycerol with a mixture of long or, preferably, medium chain fatty acids. Mixtures of fatty acids may be obtained by extraction from natural products, for example from a natural oil such as palm oil. Fatty acids extracted from palm oil contain approximately 50 to 80% by weight decanoic acid and from 20 to 50% by weight of octanoic acid. The use of a mixture of fatty acids to esterify glycerol gives a mixture of glycerides containing a mixture of different acyl chain lengths. Long and medium chain triglycerides are commercially available. For example, a preferred medium chain triglyceride (MCT) containing acyl groups with 8 to 12, more preferably 8 to 10 carbon atoms is prepared by esterification of glycerol with fatty acids extracted from palm oil, giving a mixture of triglycerides containing acyl groups with 8 to 12, more preferably 8 to 10 carbon atoms. This MCT is commercially available as Miglyol 812N (Sasol, Germany). Other commercially available MCT's include Miglyol 810 and Miglyol 818 (Sasol, Germany). A further suitable medium chain triglyceride is trilaurine (glycerol trilaurate). Commercially available long chain trigylcerides include soya bean oil, sesame oil, sunflower oil, castor oil or rape-seed oil.

Mono and di-glycerides may be obtained by partial esterification of glycerol with a suitable fatty acid, or mixture of fatty acids. If necessary the mono- and di-glycerides may be separated and purified using conventional techniques, for example by extraction from a reaction mixture following esterification. When a mono-glyceride is used it is preferably a long-chain mono glyceride, for example a mono glyceride formed by esterification of glycerol with a fatty acid containing 18 carbon atoms;

(ii) a fatty acid mono- or (preferably) di-ester of a C₂₋₁₀ diol. Preferably the diol is an aliphatic diol which may be saturated or unsaturated, for example a C₂₋₁₀-alkane diol which may be a straight chain or branched chain diol. More preferably the diol is a C₂₋₆-alkane diol which may be a straight chain or branched chain, for example ethylene glycol or propylene glycol. Suitable fatty acids include medium and long chain fatty acids described above in relation to the glycerides. Preferred esters are di-esters of propylene glycol with one or more fatty acids containing from 8 to 10 carbon atoms, for example Miglyol 840 (Sasol, Germany);

(iii) a fatty acid ester of an alkanol or a cycloalkanol. Suitable alkanols include C₁₋₁₀-alkanols, more preferably C₂₋₆-alkanols which may be straight chain or branched chain, for example ethanol, propanol, isopropanol, n-butanol, sec-butanol or tert-butanol. Suitable cycloalkanols include C₃₋₆-cycloalkanols, for example cyclohexanol. Suitable fatty acids include medium and long chain fatty acids described above in relation to the glycerides. Preferred esters are esters of a C₂₋₆-alkanol with one or more fatty acids containing from 8 to 10 carbon atoms, or more preferably 12 to 29 carbon atoms, which fatty acid may be saturated or unsaturated. Suitable esters include, for example isopropyl myristate or ethyl oleate;

(iv) a wax. Suitable waxes include esters of a long chain fatty acid with an alcohol containing at least 12 carbon atoms. The alcohol may be an aliphatic alcohol, an aromatic alcohol, an alcohol containing aliphatic and aromatic groups or a mixture of two or more such alcohols. When the alcohol is an aliphatic alcohol, it may be saturated or unsaturated. The aliphatic alcohol may be straight chain, branched chain or cyclic. Suitable aliphatic alcohols include those containing more than 12 carbon atoms, preferably more than 14 carbon atoms especially more than 18 carbon atoms, for example from 12 to 40, more preferably 14 to 36 and especially from 18 to 34 carbon atoms. Suitable long chain fatty acids include those described above in relation to the glycerides, preferably those containing more than 14 carbon atoms especially more than 18 carbon atoms, for example from 14 to 40, more preferably 14 to 36 and especially from 18 to 34 carbon atoms. The wax may be a natural wax, for example bees wax, a wax derived from plant material, or a synthetic wax prepared by esterification of a fatty acid and a long chain alcohol. Other suitable waxes include petroleum waxes such as a paraffin wax;

(v) a long chain aliphatic alcohol. Suitable alcohols include those with 6 or more carbon atoms, more preferably 8 or more carbon atoms, such as 12 or more carbon atoms, for example from 12 to 30, for example from 14 to 20 carbon atoms. It is especially preferred that the long chain aliphatic alcohol has from 6 to 20, more especially from 6 to 14 carbon atoms, for example from 8 to 12 carbon atoms. The alcohol may be straight chain, branched chain, saturated or unsaturated. Examples of suitable long chain alcohols include, 1-hexanol, 1-decanol, 1-hexadecanol, 1-octadecanol, or 1-heptadecanol (more preferably 1-decanol); or

(vi) a hydrogenated vegetable oil, for example hydrogenated castor oil.

In one embodiment of the present invention the inhibitor is selected from a medium chain triglyceride and a long chain aliphatic alcohol containing from 6 to 12, preferably from 10 to 20 carbon atoms. Preferred medium chain triglycerides and long chain aliphatic alcohols are as defined above. In a preferred embodiment the inhibitor is selected from a medium chain triglyceride containing acyl groups with from 8 to 12 carbon atoms or a mixture of such triglycerides (preferably Miglyol 812N) and an aliphatic alcohol containing from 10 to 14 carbon atoms (preferably 1-decanol) or a mixture thereof (for example a mixture comprising Miglyol 812N and 1-decanol).

Suitable, the inhibitor is liquid at ambient temperature (25° C.). When the substantially water-insoluble substance is a pharmacologically active compound the inhibitor is preferably a pharmaceutically inert material. The quantity of inhibitor in the particles is sufficient to prevent Ostwald ripening of the particles in the suspension. Preferably the inhibitor will be the minor component in the amorphous particles formed in the present process comprising the inhibitor and the substantially water-insoluble substance. Preferably, therefore, the inhibitor is present in a quantity that is just sufficient to prevent Ostwald ripening and to reduce the crystallisation rate to an acceptable level.

Suitable, the inhibitor is compatible with the substantially water-insoluble substance, i.e the water-insoluble substance in its amorphous phase is miscible with the inhibitor. One way to define miscibility of a water-insoluble substance and an inhibitor in the solid particles obtained by the present process is by the interaction parameter χ for the mixture of substance and inhibitor. Generally, the amorphous state of the substantially water-insoluble substance is suitable fully miscible with the inhibitor. Without being bound by theory, this can be defined in the Bragg-Williams theory by the parameter χ being lower than 2.

The χ parameter may be derived from the well known Bragg-Williams or the Regular Solution theories (see e.g. Jönsson, B. Lindman, K. Holmberg, B. Kronberg, “Surfactants and Polymers in Solution”, John Wiley & Sons, 1998 and Neau et al, Pharmaceutical Research, 14, 601 1997). In an ideal mixture χ is 0, and according to the Bragg-Williams theory a two-component mixture will not phase separate provided χ<2.

As disclosed in WO 03/013272, when χ is equal or less than 2.5, concentrated particle dispersions that exhibit little or no Ostwald ripening, can be prepared. Those systems in which χ is larger than about 2.5 are thought to be prone to phase separation and are less stable against Ostwald ripening. Suitably the χ value of the substance-inhibitor mixture is 2 or less, for example from 0 to 2, preferably 0.1 to 2, such as 0.2 to 1.8. However, the method of the present invention will not be bound by this theory.

Many small molecule organic substances (Mw<1000) are available in a crystalline form or can be prepared in crystalline form using conventional techniques (for example by recrystallisation from a suitable solvent system). In such cases the χ parameter of the substance and inhibitor mixture is easily determined from Equation I:

$$χ = \frac{- Δ {S}_{m} ln [ {T}_{m} / T ] / R - ln {x}_{1}^{s}}{{( 1 - {x}_{1}^{s} )}^{2}} \qquad Equation I$$

wherein:

ΔSₘ is the entropy of melting of the crystalline substantially water-insoluble substance (measured using a conventional technique such as DSC measurement);

Tₘ is the melting point (K) of the crystalline substantially water-insoluble substance (measured using a conventional technique such as DSC measurement);

T is the temperature at the solubility experiment

R is the gas constant; and

x^{s}₁ is the mole fraction solubility of the crystalline substantially water-insoluble substance in the inhibitor (measured using conventional techniques for determining solubility for example as hereinbefore described). In the above equation Tₘ and ΔSₘ refer to the melting point of the crystalline form of the material. In those cases where the substance may exist in the form of different polymorphs, Tₘ and ΔSₘ are determined for the polymorphic form of the substance that is used in the solubility experiment. As will be understood, the measurement of ΔSₘ, Tₘ and x^{s}₁ are performed on the crystalline substantially water-insoluble substance prior to formation of the dispersion according to the invention and thereby enables a preferred inhibitor for the substantially water-insoluble material to be selected by performing simple measurements on the bulk crystalline material.

The mole fraction solubility of the crystalline substantially water-insoluble substance in the inhibitor (x^{s}₁) is simply the number of moles of substance per mole of inhibitor present in a saturated solution of the substance in the inhibitor. As will be realized the equation above is derived for a two component system of a substance and an inhibitor. In those systems where the inhibitor contains more than one compound (for example in the case of a medium chain triglyceride comprising a mixture of triglycerides such as Miglyol 812N, or where a mixture of inhibitors is used) it is sufficient to calculate x^{s}₁ in terms of the “apparent molarity” of the mixture of inhibitors.

The apparent molarity of such a mixture is calculated for a mixture of inhibitor components to be:

Apparent molarity=Mass of 1 litre of inhibitor mixture*[(a/Mwa)+(b/Mwb)+. . . (n/Mwn)]

wherein:

a, b . . . n are the weight fraction of each component in the inhibitor mixture (for example for component a this is % w/w component a/100); and

Mwa . . . Mwn is the molecular weight of each component a . . . n in the mixture.

x^{s}₁ is then calculated as:

$${x}_{1}^{s} = \frac{\begin{matrix}Molar solubility of the crystalline \\ substance in the inhibitor mixture ( mol \text{/} 1 )\end{matrix}}{Apparent molarity of inhibitor mixture ( mol \text{/} 1 )}$$

When the inhibitor is a solid at the temperature that the dispersion is prepared, the mole fraction solubility, x^{s}₁, can be estimated by measuring the mole fraction solubility at a series of temperatures above the melting point of the inhibitor and extrapolating the solubility back to the desired temperature. However, as hereinbefore mentioned, it is preferred that the inhibitor is a liquid at the temperature that the dispersion is prepared. This is advantageous because, amongst other things, the use of a liquid inhibitor enables the value of x^{s}₁ to be measured directly.

In certain cases, it may not be possible to obtain the substantially water-insoluble material in a crystalline form, particularly in the case of large organic molecules which may be amorphous. In such cases, preferred inhibitors are those which are sufficiently miscible with the substantially water-insoluble material to form a substantially single phase mixture (according to the theory above, χ<2) when mixed in the required substance:inhibitor ratio. Miscibility of the inhibitor in the substantially water-insoluble material may be determined using routine experimentation. For example the substance and inhibitor may be dissolved in a suitable organic solvent followed by removal of the solvent to leave a mixture of the substance and inhibitor. The resulting mixture may then be characterised using a routine technique such as DSC characterisation to determine whether or not the mixture is a single phase system. This empirical method enables preferred inhibitors for a particular substance to be selected and will provide substantially single phase particles in the dispersion prepared according to the present invention.

The Co-Inhibitor

In a further embodiment of the present invention a suitable co-inhibitor is present in the first solution in the present process. In those cases, the inhibitor is treated as a pseudo-one component mixture. The presence of the co-inhibitor increases the miscibility of the substance and the inhibitor mixture, thereby reducing the χ value and further reducing or preventing Ostwald ripening. Suitable co-inhibitors include an inhibitor as hereinbefore is defined, preferably an inhibitor selected from classes (i) to (vi) listed hereinbefore. In a preferred embodiment when the inhibitor is a medium chain triglyceride containing acyl groups with 8 to 12 carbon atoms (or a mixture of such triglycerides such as Miglyol 812N), a preferred co-inhibitor is a long chain aliphatic alcohol containing 6 or more carbon atoms (preferably from 6 to 14 carbon atoms) for example 1-hexanol or more preferably 1-decanol. Other suitable co-inhibitors include hydrophobic polymers, for example polypropylene glycol 2000, and hydrophobic block copolymers, for example the tri-block copolymer Pluronic L121. The weight ratio of inhibitor:co-inhibitor is selected to give the desired χ value of the mixture of the substance and the inhibitor (mixture) and may be varied over wide limits, for example from 10:1 to 1:10 (w/w), for example 1:2 (w/w) and approximately 1:1 (w/w). Preferred values for χ are as hereinbefore defined.

In one embodiment of the present invention a stable dispersion of particles of a substantially water-insoluble pharmacologically active substance in an aqueous medium is provided. The dispersions prepared according to this embodiment exhibit little or no growth in particle size during storage resulting from Ostwald ripening.

In one embodiment it is preferred that the miscibility of the substantially water-insoluble substance and inhibitor are sufficient to give substantially single phase particles in the dispersion, more preferably the inhibitor/substance mixture has a χ value of <2.5, more preferably 2 or less, for example from 0 to 2 wherein the χ value is as hereinbefore defined.

In one embodiment the inhibitor is preferably a medium chain tri-glyceride (MCT) containing acyl groups with 8 to 12 carbon atoms, more preferably 8 to 10 carbon atoms, or a mixture thereof, for example Miglyol 812N. The miscibility of the inhibitor with the substance may be increased by using a co-inhibitor as hereinbefore described. For example, a suitable inhibitor/co-inhibitor in this embodiment comprises a medium chain tri-glyceride (MCT) as defined above and a long chain aliphatic alcohol having 6 to 12, more preferably 8 to 12, for example 10, carbon atoms, or a mixture comprising two or more such inhibitors, for example 1-hexanol or, more preferably, 1-decanol. A preferred mixture of inhibitor/co-inhibitor for use in this embodiment is a mixture of Miglyol 812N and 1-decanol.

If required the particles present in the dispersion prepared according to the present invention may be isolated from the aqueous medium. The particles may be separated using conventional techniques, for example by centrifuging, reverse osmosis, membrane filtration, lyophilisation or spray drying. Isolation of the particles is useful because it allows the particles to be washed and re-suspended in a sterile aqueous medium to give a suspension suitable for administration to a warm blooded mammal, especially a human, for example by oral or parenteral e.g. intravenous, administration.

In one embodiment an agent may be added to the suspension prior to isolation of the particles to prevent agglomeration of the solid particles during isolation, for example spray-drying, spray-granulation or lyophilisation. Suitable agents include for example a sugar, such as mannitol.

Isolation of the particles from the suspension is also useful when it is desirable to store the particles as a powder. The powder may then be re-suspended in an aqueous medium prior to use. This is particularly useful when the substantially water-insoluble substance is a pharmacologically active substance. The isolated particles of the substance may then be stored as a powder in, for example, a vial and subsequently be re-suspended in a suitable liquid medium for administration to a patient as described above.

Alternatively the isolated particles may be used to prepare solid formulations, for example by blending the particles with suitable excipients/carriers and granulating or compressing the resulting mixture to form a tablet or granules suitable for oral administration. Alternatively the particles may be suspended, dispersed or encapsulated in a suitable matrix system, for example a biocompatible polymeric matrix, for example a hydroxypropyl methylcellulose (HPMC) or polylactide-co-glycloide polymer to give a controlled or sustained release formulation.

In another embodiment of the present invention the process may be performed at such high temperatures, that a sterile dispersion is provided directly, and which dispersion can be administered to a warm blooded mammal as described above without the need for additional purification or sterilisation steps.

According to a further aspect of the present invention a stable aqueous dispersion is provided comprising a continuous aqueous phase in which particles are dispersed. These dispersed particles comprise an inhibitor and a substantially water-insoluble substance, and the said dispersion is obtainable by the process according to the present invention; and wherein:
(i) the inhibitor is a compound that is substantially insoluble in water;
(ii) the inhibitor is less soluble in water than the substantially water-insoluble substance; and
(iii) the inhibitor is completely miscible with the amorphous phase of the substantially water-insoluble substance.

The dispersion according to this aspect of the present invention exhibit little or no particle growth upon storage, mediated by Ostwald ripening (i.e. the dispersion is a stable dispersion as defined above), and reduced crystallization rate of the amorphous sub-micron particle.

The particles preferably have a mean diameter of less than 1 μm and more preferably less than 500 nm. It is especially preferred that the particles in the dispersion have a mean particle size of from 10 to 500 nm, more especially from 50 to 300 nm and still more especially from 100 to 200 nm.

The particles may contain a single substantially water-insoluble substance, or two or more of such substances. The particles may contain a single inhibitor or a combination of an inhibitor and one or more co-inhibitors as hereinbefore described.

Medical Use

When the substance is a substantially water-insoluble pharmacologically active material, the dispersions according to the present invention may be administered to a warm blooded mammal (especially a human), for example by oral or parenteral (e.g. intravenous) administration. In an alternative embodiment the dispersion may be used as a granulation liquid in a wet granulation process to prepare granules comprising the substantially water-insoluble pharmacologically active material and one or more excipients, optionally after first concentrating the dispersion by removal of excess aqueous medium. The resulting granules may then be used directly, for example by filling into capsules to provide a unit dosage containing the granules. Alternatively the granules may be optionally mixed with further excipients, disintegrants, binders, lubricants etc. and compressed into a tablet suitable for oral administration. If required the tablet may be coated to provide control over the release properties of the tablet or to protect it against degradation, for example through exposure to light and/or moisture. Wet granulation techniques and excipients suitable for use in tablet formulations are well known in the art.

According to a further aspect of the present invention there is provided a solid particle comprising an inhibitor and a substantially water-insoluble substance obtainable by the process according to the present invention, wherein the substance and the inhibitor are as hereinbefore defined.

Preferred particles are those described herein in relation to the dispersions according to the present invention, especially those in which the substantially water-insoluble substance is a substantially water-insoluble pharmacologically active substance, for example as described herein.

According to a further aspect of the present invention there is provided a solid particle comprising an inhibitor and a substantially water-insoluble pharmacologically active substance obtainable by the process according to the present invention, for use as a medicament, wherein the substance and the inhibitor are as hereinbefore defined.

According to a further aspect of the present invention there is provided a pharmaceutical composition comprising a pharmaceutically acceptable carrier or diluent in association with a solid particle comprising an inhibitor and a substantially water-insoluble pharmacologically active substance obtainable by the process according to the present invention.

Suitable pharmaceutically acceptable carriers or diluents are well known excipients used in the preparation of pharmaceutical formulations, for example, fillers, binders, lubricants, disintegrants and/or release controlling/modifying excipients.

The invention is further illustrated by the following examples in which all parts are parts by weight unless stated otherwise.

### EXAMPLES

A light scattering method according to the following was used in the following examples for determination of bulk concentrations in amorphous sub-micron dispersions:

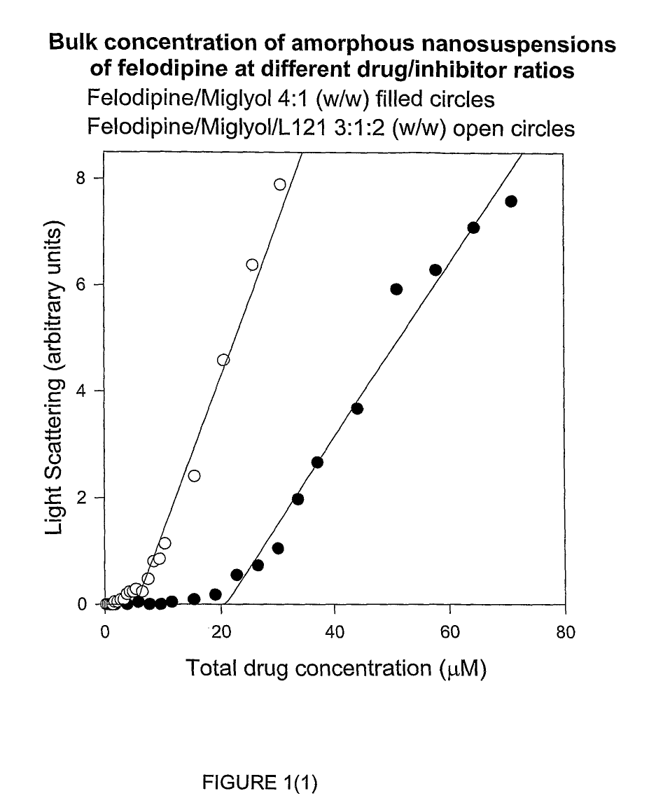

The amorphous solubility, i.e. the bulk concentration in amorphous submicron dispersion, was measured by adding small volumes of drug suspension successively to a fluorescence cuvette containing pure liquid and mixed to give the desired concentrations. The light scattering intensity at 700 nm was recorded at a scattering angle of 90° as a function of total drug concentration. As a light scattering setup a Perkin Elmer LS 55 Luminiscence Spectrometer was used, setting both the emission and excitation wave lengths to 700 nm (Mougán, M. A. et al., Journal of Chemical Education., 72, 284 (1995)). The solubility was determined from a plot of light scattering intensity vs. concentration of drug, as the onset of a linear increase in the scattering intensity. In FIG. 1, results are shown from measurements of the bulk concentrations (amorphous solubility) in amorphous submicron dispersion of felodipine for different felodipine/inhibitor ratios (w/w) as used in Examples 1a and 1b.

### Example 1a

### 10% Felodipine Amorphous Submicron Dispersion (Felodipine/Miglyol 4:1 (w/w)

An oil-in-water emulsion containing 10% (w/w) Miglyol 812N, 0.45% (w/w) polyvinyl pyrrolidone K30 (PVP) and 0.18% (w/w) sodium dodecylsulphate (SDS) was prepared using sonication for 60 minutes (Elma Transonic Bath T460). The emulsion droplet size was measured using dynamic light scattering (Brookhaven Fiber-Optic Quasi-Elastic Light Scattering; FOQELS) to 195 nm.

A 20% (w/w) suspension of crystalline felodipine in water containing 0.32% (w/w) SDS was prepared by sonication and stirring, having a volume-averaged particle size of 13.4 μm, as measured by laser diffraction (Malvern Mastersizer 2000). 0.25 mL of the emulsion was mixed with 0.25 mL water and 0.5 mL of the suspension and heated in high-pressure vials (Biotage, Sweden) to 155° C. for 10 minutes under magnetic stirring at 300 rpm. The mixture was then cooled down to room temperature without stirring and the particle size measured with dynamic light scattering to 250 nm.

After 3 hours of storage at room temperature, crystals appeared on the bottom of the vials and after approximately 1 day the whole suspension was crystalline.

### Example 1b

### 10% Felodipine Amorphous Submicron Dispersion (Felodipine/Miglyol/L121 3:1:2 (w/w/w)

An oil-in-water emulsion containing 20% (w/w) Miglyol 812N/Pluronic L121 (1:2 w/w) and 0.57% (w/w) sodium dodecyl sulphate (SDS) was prepared as follows; an oil-in-water emulsion containing 20% (w/w) Miglyol 812N and 1.7% (w/w) sodium dodecyl sulphate (SDS) was prepared using a Polytron homogenizer followed by high-pressure homogenization (Rannie). To this emulsion the co-inhibitor Pluronic L121 and water was added and mixed by stirring at approximately 0° C. for 1 h, interrupted by 3×5 minutes sonication, giving a final emulsion containing 6.7% (w/w) Miglyol 812N, 13.3% (w/w) PluronicL121 and 0.57% (w/w) SDS. The emulsion droplet size was measured using dynamic light scattering to 120 nm.

A 20% (w/w) suspension of crystalline felodipine in water containing 0.32% (w/w) SDS was prepared by sonication and stirring, having a volume-averaged particle size of 13.4 μm, as measured by laser diffraction. 0.5 mL of the emulsion was mixed with 0.5 mL of the suspension and heated in high-pressure vials 155° C. for 10 minutes. The mixture was then cooled down to room temperature and the particle size measured with dynamic light scattering to 135 nm.

After 2 weeks of storage at room temperature no crystals were visible in the nanosuspension, i.e. a significant reduction of the crystallization rate.

### Example 2a

### 10% Fenofibrate Amorphous Submicron Dispersion (Fenofibrate/Miglyol 4:1 (w/w)

An oil-in-water emulsion containing 10% (w/w) Miglyol 812N, 0.4% sodium dodecyl sulphate (SDS) and 10 mM NaCl was prepared using sonication for 60 minutes. The emulsion droplet size was measured using dynamic light scattering to 160 nm.

A 20% (w/w) suspension of crystalline fenofibrate in water containing 1.6% (w/w) polyvinyl pyrrolidone K30 (PVP) and 0.32% SDS was prepared by sonication and stirring, having a volume-averaged particle size of 10.0 μm, as measured by laser diffraction. 0.25 mL of the emulsion was mixed with 0.25 ml H₂O and 0.5 mL of the suspension and heated in an ordinary glass vial to 100° C. for 10 minutes. The mixture was then cooled down to room temperature and the particle size measured with dynamic light scattering to 204 nm.

After 2 hours of storage at room temperature, crystals appeared on the bottom of the vial and after approximately 2 days the whole suspension was crystalline.

### Example 2b

### 10% Fenofibrate Amorphous Submicron Dispersion (Fenofibrate/Miglyol 2:1 (w/w)

An oil-in-water emulsion containing 10% (w/w) Miglyol 812N, 0.4% sodium dodecyl sulphate (SDS) and 10 mM NaCl was prepared using sonication for 60 minutes. The emulsion droplet size was measured using dynamic light scattering to 160 nm.

A 20% (w/w) suspension of crystalline fenofibrate in water containing 1.6% (w/w) polyvinyl pyrrolidone K30 (PVP) and 0.32% SDS was prepared by sonication and stirring, having a volume-averaged particle size of 10.0 μm, as measured by laser diffraction. 0.5 mL of the emulsion was mixed with 0.5 mL of the suspension and heated in an ordinary glass vial to 100° C. for 10 minutes. The mixture was then cooled down to room temperature and the particle size measured with dynamic light scattering to 190 nm.

After 2 weeks of storage at room temperature no crystals were visible in the submicron dispersion.

### Example 3a

### 10% Triclosan Amorphous Submicron Dispersion (Triclosan/Miglyol 4:1 (w/w)

An oil-in-water emulsion containing 5% (w/w) Miglyol 812N, 0.2% (w/w) sodium dodecyl sulphate (SDS) and 5 mM NaCl was prepared using sonication for 60 minutes. The emulsion droplet size was measured using dynamic light scattering to 185 nm.

A 20% (w/w) suspension of crystalline triclosan in water containing 0.32% (w/w) SDS was prepared by sonication and stirring, having a volume-averaged particle size of 92 μm, as measured by laser diffraction. 0.5 mL of the emulsion was mixed with 0.5 mL of the suspension and heated in an ordinary glass vial to 100° C. for 10 minutes. The mixture was then cooled down to room temperature and the particle size measured with dynamic light scattering to 200 nm.

After 2 hours of storage at room temperature, crystals appeared on the bottom of the vials and after approximately 1 day the whole suspension was crystalline.

### Example 3b

### 10% Triclosan Amorphous Submicron Dispersion (Triclosan/Miglyol 2:1 (w/w)

An oil-in-water emulsion containing 10% (w/w) Miglyol 812N, 0.4% (w/w) sodium dodecyl sulphate (SDS) and 10 mM NaCl was prepared using sonication for 60 minutes. The emulsion droplet size was measured using dynamic light scattering to 185 nm.

A 20% (w/w) suspension of crystalline triclosan in water containing 0.32% (w/w) SDS was prepared by sonication and stirring, having a volume-averaged particle size of 92 μm, as measured by laser diffraction. 0.5 mL of the emulsion was mixed with 0.5 mL of the suspension and heated in an ordinary glass vial to 100° C. for 10 minutes. The mixture was then cooled down to room temperature and the particle size measured with dynamic light scattering to 185 nm.

After 2 weeks of storage at room temperature no crystals were visible in the submicron dispersion

1. A process for the preparation of a stable dispersion of solid amorphous submicron particles in an aqueous medium comprising:
1) combining
a) an emulsion comprising
a continuous aqueous phase;
an inhibitor; and
a stabiliser;
with
b) the substantially a substantially water-insoluble substance; wherein the ratio of substantially water-insoluble substance to inhibitor is below 10:1 (w/w); and
2) increasing the temperature to vicinity of the melting temperature of the substantially water-insoluble substance.

2. A process according to claim 1 wherein the substantially water-insoluble substance is in its crystalline state.

3. A process according to claim 1 wherein the substantially water-insoluble substance is amorphous.

4. A process according to claim 1 wherein the substantially water-insoluble substance in its crystalline state is added as a suspension.

5. A process according to claim 1 wherein the substantially water-insoluble substance is a substantially water-insoluble pharmacologically active compound.

6. A process according to claim 1 wherein the melting point of the water insoluble substance is below 300° C.

7. A process according to claim 1 wherein the melting point of the water insoluble substance is equal to or below 225° C.

8. A process according to claim 1 wherein the melting point of the water insoluble substance is equal to or below 200° C.

9. A process according to claim 1 wherein the melting point of the water insoluble substance is equal to or below 175° C.

10. A process according to claim 1 wherein the aqueous medium consists of water.

11. A process according to claim 1 wherein step 2) is performed under high pressure.

12. A process according to claim 1 wherein the inhibitor is sufficiently miscible with the substantially water-insoluble material to form solid particles in the dispersion comprising a substantially single phase mixture of the substance and the inhibitor.

13. A process according to claim 1 wherein the inhibitor is a mixture of triglycerides obtainable by esterifying glycerol with a mixture of medium chain fatty acids.

14. A process according to claim 1 wherein the inhibitor is selected from mono-, di- or triglyceride of a fatty acid, fatty acid mono- or di-ester of a C₂₋₁₀ diol, a fatty acid ester of an alkanol or a cycloalkanol, a wax, a long chain aliphatic alcohol or a hydrogenated vegetable oil, or a combination of two or more inhibitors.

15. A process according to claim 12 wherein the inhibitor is selected from medium chain triglycerides containing acyl groups with 8 to 12 carbon atoms.

16. A process according to claim 13 wherein the inhibitor is selected from Miglyol 810N, Miglyol 812N, and Miglyol 818N.

17. A process according to claim 1 wherein the inhibitor is Miglyol 812N.

18. A process according to claim 1 wherein the ratio of water insoluble substance and inhibitor is 2:1 w/w by weight.

19. A process according to claim 1 wherein the ratio of water-insoluble substance and inhibitor is 1:1 w/w by weight.

20. A process according to claim 1 wherein the emulsion in step 1a) further contains a co-inhibitor.

21. A process according to claim 20 wherein the co-inhibitor is selected from mono-, di- or triglyceride of a fatty acid, fatty acid mono- or di-ester of a C₂₋₁₀ diol, fatty acid ester of an alkanol or a cycloalkanol, wax, a long chain aliphatic alcohol or a hydrogenated vegetable oil.

22. A process according to claim 20 wherein the co-inhibitor is selected from a medium chain triglyceride containing acyl groups with 8 to 12 carbon atoms, a long chain aliphatic alcohol containing 6 to 14 carbon atoms, polypropylene glycol 2000, and a hydrophobic block copolymer.

23. A process according to claim 20 wherein the co-inhibitor is selected from Miglyol 812N, 1-hexanol and 1-decanol.

24. A process according to claim 1 further comprising isolating the solid particles from the dispersion.

25. A process according to claim 1 wherein the temperature is increased to a temperature of ±20° C. of the melting temperature of the active substance.

26. A process according to claim 4 wherein a stabiliser is added to the suspension.

27. A process according to claim 1 wherein the stabiliser is a polymeric dispersant or a surfactant, or a mixture thereof.

28. A process according to claim 1 wherein the aqueous phase comprises a stabiliser in amount of 0.01 to 10% by weight.

29. A dispersion of amorphous submicron particles, obtainable by the process according to claim 1.

30. (canceled)

31. A pharmaceutical composition comprising the dispersion according to claim 29 in association with a pharmaceutically acceptable carrier of diluent.
